(19) United States
(12) Patent Application Publication
(10) Pub. No.: US 2010/0285555 A1
(43) Pub. Date: Nov. 11, 2010

(54) DISTRIBUTED BIOFUEL MANUFACTURING SYSTEM (DBMS)

(75) Inventors: JERRY B. WARNER, Alexandria, VA (US); Christopher Michael Haggerson, Arlington, VA (US); Michael Ralph Ladisch, West Lafayette, IN (US); Nathan Scott Moster, West Lafayette, IN (US)

Correspondence Address: Raj Abhyanker, P.C., 1580 West, El Camino Real, Suite 8, Mountain View, CA, 94040, US

(73) Assignee: Defense Life Sciences, LLC, McLean, VA (US)

(21) Appl. No.: 12/772,260

(22) Filed: May 3, 2010

Related U.S. Application Data
(60) Provisional application No. 61/215,521, filed on May 7, 2009.

(51) Int. Cl. C12P 7/10; C12M 1/00

(52) U.S. Cl. 435/165; 435/289.1

(57) ABSTRACT

The Distributed Biofuel Manufacturing System (DBMS) integrates three critical innovations which may advance the state of the art for biofuels manufacturing to a distributed production and local distribution model. This model yields a sustainable and commercially viable means of production for biofuels. Essential to this new model is a Portable Biorefinery (PBR) which allows for onsite conversion of biomass into biofuels, an accompanying set of advanced biocatalysts, and finally a multi-role harvesting and pre-processing technology to optimize both deliberate and “threat” biomass for biofuels. Utilizing either high yield deliberate biomass “plantations” or salvaging available threat biomass, the portable biorefinery will process high volumes of variable biomass into dense liquid energy format and other energy products which can then be utilized locally. This disclosure seeks to avoid the logistical overhead and economic limitations of traditional centralized industrial fuels manufacturing.

### CLAIM OF PRIORITY

This application claims priority from a U.S. Provisional Application No. 61/215,521 filed on May 7, 2009 titled: DISTRIBUTED BIOFUEL MANUFACTURING SYSTEM (DBMS).

### FIELD OF TECHNOLOGY

This disclosure relates generally to a technical field of process manufacturing and, in one embodiment, to a method, system and apparatus for Distributed Biofuel Manufacturing System (DBMS).

### BACKGROUND

Previous approaches to producing cellulosic biofuels have almost always centered on the development of fixed, large-scale bio-refineries where the feedstock materials have been transported to the site, converted, and then entered into the commercial fuel system for market.

This model of production, even with non-cellulosic feedstock such as corn and sugar cane, has required government intervention and subsidies to be commercially viable in the past.

### SUMMARY

The disclosure also known as the Distributed Biofuel Manufacturing System (DBMS), integrates three critical innovations that may advance the state of the art for biofuels manufacturing to a distributed production and local distribution model. This model may yield a sustainable and commercially viable means of production for biofuels. It contains a Portable Bio-refinery (PBR) that may allow for onsite conversion of biomass into biofuels, an accompanying set of advanced biocatalysts, and finally a multi-role harvesting and pre-processing technology to optimize both deliberate and “threat” biomass for biofuels. Utilizing either high yield deliberate biomass “plantations” or salvaging available threat biomass, the portable bio-refinery may process high volumes of variable biomass into dense liquid energy format and other energy products which can then be utilized locally. This disclosure seeks to avoid the logistical overhead and economic limitations of traditional centralized industrial fuels manufacturing.

As an example, using the disclosed system and apparatus, federal installations may allocate as little as 60 acres of land approximately, where hybrid poplar or other high-yield, non-crop biomass would be cultivated. In the case of the hybrid poplar, each acre may yield approximately 800-1,000 gallons of biofuel annually, with the possible 60 acre plantation able to provide sufficient E-85 fuel for a fleet of approximately 55 E-85 vehicles.

In the case of “threat” biomass that may include infected pine, infected ash or Tamarisk trees along drainages, the DBMS/mobile bio-refinery may be used to create defensive zones to protect critical infrastructure and water resources by possible means of harvesting infected trees and consequently may provide liquid fuel and wood fuel pellets to the local economy.

### BRIEF DESCRIPTION OF THE VIEWS OF DRAWINGS

Example embodiments are illustrated by way of example and not limitation in the figures of accompanying drawings, in which like references indicate similar elements and in which:

FIG. 1 is an overview of the embodiments contained and therein chemical reactions at the Distributed Biofuel Manufacturing System (DBMS) portable bio-refinery, according to one embodiment.

FIG. 2 is an overview of the process flow of the operations performed by the Distributed Biofuel Manufacturing System (DBMS) in relation to its various embodiments, according to one embodiment.

FIG. 3 illustrates a diagram that describes the operations and process that may be necessitated for the Poplar Fuel Point Application, according to one embodiment.

Drawings and from the Detailed Description are as follows.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components and with reference to symbolic representations of operations, processing tasks, and functions that may be performed by various computing components or devices. It should be appreciated that the various block components shown in the figures may be realized by any number of hardware, mechanical, and/or firmware components configured to perform the specified functions. For example, an embodiment of a system or a component may employ various integrated circuit components, e.g., memory elements, digital signal processing elements, logic elements, or the like, which may carry out a variety of functions under the control of one or more microprocessors or other control devices.

The present disclosure relates in general to the lack of commercially viable and agronomically sustainable means for producing cellulosic bio-ethanol. The difficulties of addressing this problem may be attributable to current efforts that rely on traditional centralized industrial means of production and distribution. Methods that may offer an alternative model with distributed production and local distribution have not yet been adequately developed.

Woody biomass is particularly difficult, as it often takes up to one ton of dry weight material to yield less than 100 gallons of liquid fuel. In most instances the costs of harvesting and transportation to a fixed production facility outstrip the value of the resultant product.

The availability of commercially sustainable bio-ethanol is limited, and many new flex fuel, or hybrid vehicles may not have access to E-85, or other blended biofuels substitutions for traditional petroleum based fuels.

In aggregate, many of the recently legislated requirements for carbon reduction and alternative fuels may have been constrained by adequate supply of biofuels.

Previous approaches to expanding the use of biofuels for vehicle fleets have been largely regulatory. Recently, the Department of Defense has mandated that all U.S. military installations establish an E-85 pump at each filling station; however this requirement may not significantly affect the large supply chain of U.S. fuels production.

Regulatory and potential demand from flex-fuel vehicles may not significantly affected the supply chain to increase biofuels production. Underlying this disadvantage is the current problem of the industrial biofuel production model.

As an example, using the disclosed system, federal installations may possibly allocate as little as 60 acres of land where hybrid poplar or other high-yield, non-food crop biomass may be cultivated.

Another aspect that may contribute to the lack of commercially and agronomically viable biofuels production is the land use competition. Many fuel crops mostly compete with other imperatives such as food, water use, availability of large acreage and soil depletion.

Alternatively, there may be significant acreage identified which may have become contaminated from various forms of activity and that may have been restricted to any form of use.

Limited progress may be made with decontamination of such sites due to contemporary soil remediation technologies being expensive.

Previous approaches to avoiding such conflicts may be the reason for the overall shift to cellulosic biomass and non-farming acreage for fuels, such as growing switch grass on prairie acreage. Most contaminated acreage lie fallow because the land may often contain petroleum, explosives chemistries or heavy metal content in the soil and groundwater. Remediation of these sites may often involve significant excavation, chemical and biological reduction.

Under the above mentioned circumstances and other contexts, utilization of such contaminated land for any commercial use may be severely restricted and may foresee no provision for agricultural use.

Another related problem may be the proliferation of forest infections and non-native plant species threatening U.S. forests and drainages. These could include the Lodge Pole Pine beetle infestation in the Rocky Mountains, the Emerald Ash borer in the Midwest and the Tamarisk tree among other examples.

In most circumstances these “threat” biomass resources may go unchecked and may be allowed to spread or may naturally decay. Infected acreage may be often cut and burned, that would add to carbon dioxide levels in the atmosphere. Previous treatment methods and ways to utilize the affected woody biomass have been attempted with projects that harvest and transport infected biomass to fixed production facilities for biofuel and wood pellet production.

These efforts were found problematic as the utilization of this woody biomass was mostly inflicting significant logistical overhead to transport for processing as well as increasing the danger of spreading contamination if infected biomass is transported for alternative use.

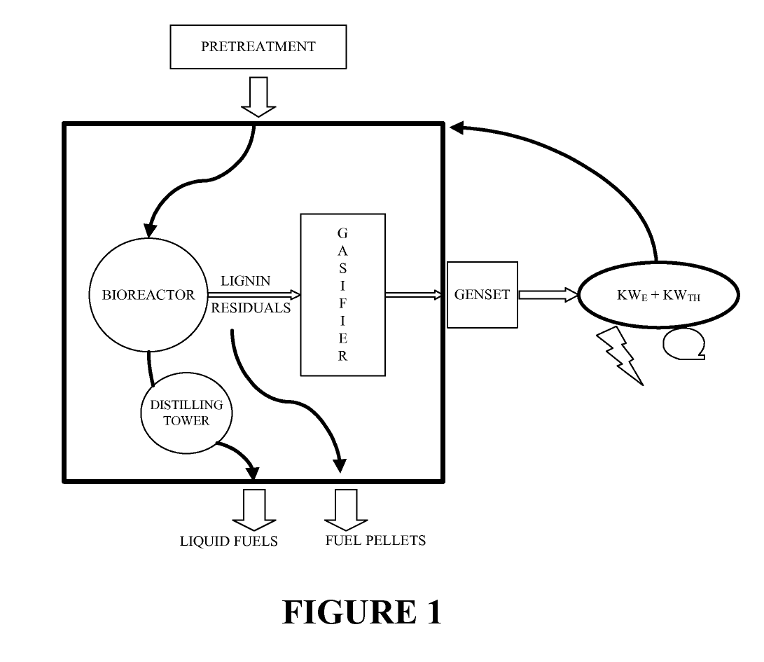

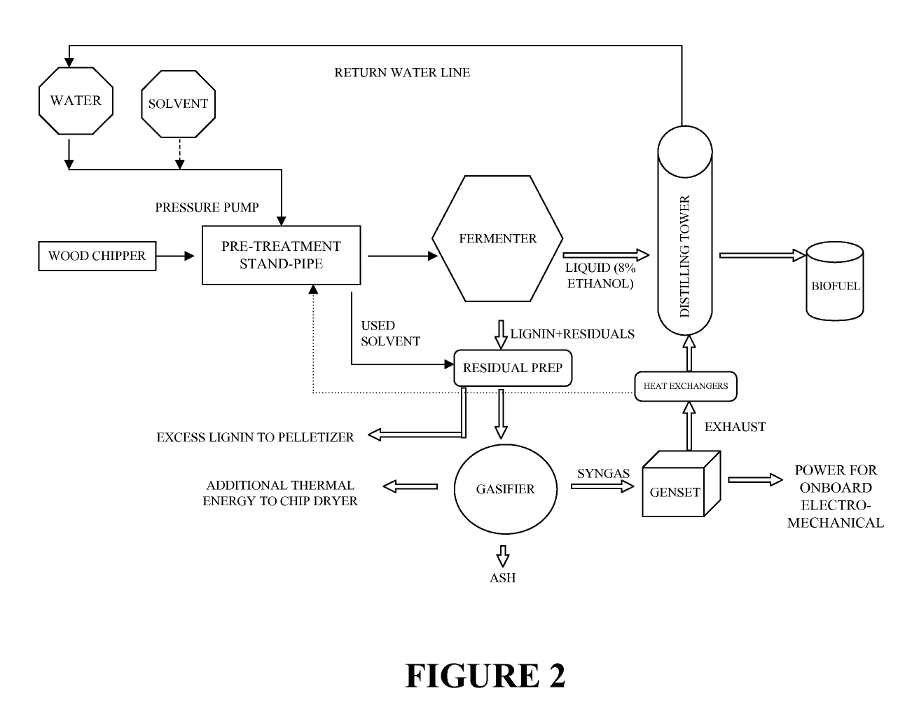

In one embodiment, the pretreatment of woody biomass is illustrated. Such woody bio-mass may be deliberately cultivated or salvaged as a possible threat to the environment. FIG. 1 embodies the DBMS portable bio-refinery, that shows the pretreatment stage of the woody bio-mass, progress to the bio-mass being subjected to the bio-reactor, that may go on to use distilling power or other process to further refine the material into biofuel. Such distilling power may be used in the DBMS portable bio-refinery to generate the woody biomass to a form of liquidized fuel. FIG. 1, also illustrates how the residuals in the DBMS bio-refinery that may contain Lignin material, be sent to the gasifier coupled with a generator system that is situated within the DBMS portable bio-refinery plant that may provide electricity, thermal and dynamic energy to other hardware components. FIG. 2 embodies the set of components that make up the process flow and operations mode of the DBMS. In FIG. 2, the water supply is indicated at the outset, after which the solvent for the bio-mass may be added to the supply line. This mixture may then proceed to be pumped using pressure into the pre-treatment stand pipe. A wood chipper shown in FIG. 2 may directly send the possible woody bio-mass to such stand pipe. The Fermenter shown in FIG. 2 may receive the pulp or bio-mass extract or the bio-mass mixture wherein an 8% of bio-fuel solution may be fermented and evacuated to the distilling tower. Once distilled in the distilling tower illustrated in FIG. 2, the process may conclude to show an output of bio-fuel.

In FIG. 2, the heat exchangers that help in the distillation process in the distilling tower may also be present in the pre-treatment stage in the stand pipe. For hardwood that may be present in the stand pipe, adjustment of pressure and temperature may be done. On the softwood, an inclusion of solvent may be done in addition to the above factors of pressure and temperature.

FIG. 2, shows that used solvent may be sent to the residual preparations that may include the residuals and lignin, from the fermenter process. Such mass may proceed to the Gasifier stage where the thermal energy that is output would be pushed to generate power to the chip dryer. The byproducts may be ash. The synthetic gas that may be generated as a result of the processes defined above in the Gasifier embodiment may be sent to the Genset that may power the electro-mechanical processes. FIG. 2 also shows how the exhaust from the Genset leads to the heat exchangers thereby making it a synchronized and low-wastage of material operational scheme.

In FIG. 2, an excess of lignin instead of being wasted may be sent to the pelletizer for the process of wood pellets.

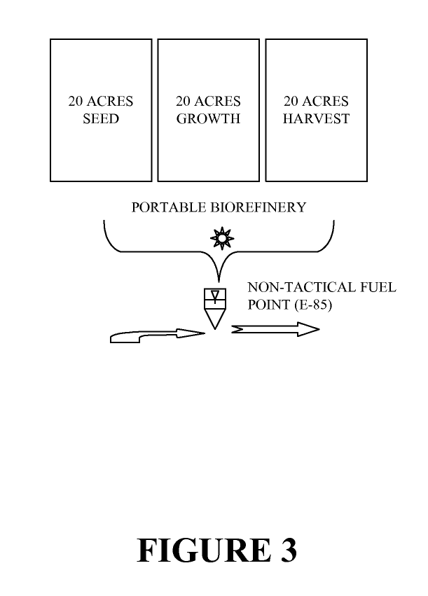

FIG. 3 embodies the probabilities of adding the DBMS system and apparatus to three situations. The First situation may be when the land, an approximate figure of 20 acres is planted. The second, may be when the land, an approximate figure of 20 acres is in growth. The third may be once the 20 acres yield the growth and is thereby harvested, the DMBS system may be placed for use of generating renewable fuel for non-tactical flex fuel vehicles.

The disclosure also known as the Distributed Biofuel Manufacturing System (DBMS), integrates three critical innovations that may advance the state of the art for biofuels manufacturing to a distributed production and local distribution model. This model may yield a sustainable and commercially viable means of production for biofuels. It contains a Portable Bio-refinery (PBR) that may allow for onsite conversion of biomass into biofuels, an accompanying set of advanced biocatalysts, and finally a multi-role harvesting and pre-processing technology to optimize both deliberate and “threat” biomass for biofuels. Utilizing either high yield deliberate biomass “plantations” or salvaging available threat biomass, the portable bio-refinery may process high volumes of variable biomass into dense liquid energy format and other energy products which can then be utilized locally. This disclosure seeks to avoid the logistical overhead and economic limitations of traditional centralized industrial fuels manufacturing.

As an example, using the disclosed system and apparatus, federal installations may allocate as little as 60 acres of land approximately, where hybrid poplar or other high-yield, non-crop biomass would be cultivated. In the case of the hybrid poplar, each acre may yield approximately 800-1,000 gallons of biofuel annually, with the possible 60 acre plantation able to provide sufficient E-85 fuel for a fleet of approximately 55 E-85 vehicles.

In the case of “threat” biomass that may include infected pine, infected ash or Tamarisk trees along drainages, the DBMS/mobile bio-refinery may be used to create defensive zones to protect critical infrastructure and water resources by possible means of harvesting infected trees and consequently may provide liquid fuel and wood fuel pellets to the local economy.

These embodiments may use hybrid poplars, tuned bio-catalysts for specific examples, however the apparatus and system may be used on a universal basis.

The system and all of its embodiments refer to;

Distributed Biofuel Manufacturing System (DBMS) that defines an integrated system with an apparatus to convert deliberate or threat biomass utilizing a portable biorefinery, co-developed catalysts and onsite biofuel and wood pellet production.

Such embodiment may be further defined as an integrated system utilizing plantations of hybrid or transgenic poplar trees to produce biofuels and wood pellets while performing decontamination of soil or ground water.

The DBMS contains a Portable Bio-Refinery (PBR) apparatus that may integrate biocatalytic and thermochemical technologies to convert woody biomass to products while utilizing the residuals to produce thermal, dynamic and electrical power for the entire process.

A method and system is defined that remediates threat biomass by onsite conversion of threat biomass into usable commercial products not limited to the usage of; Duel Use Pre-treatment Equipment an apparatus including a pre-treatment standpipe which can prepare either hard or softwood biomass for conversion to biofuels.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the disclosure, which includes known equivalents and foreseeable equivalents at the time of filing this patent application.

1. A system, comprising:
an integrated system with an apparatus to convert a biomass utilizing a portable bio-refinery, co-developed catalysts and onsite biofuel and wood pellet production.

2. The system of claim 1 further comprising:
a Portable Bio-Refinery (PBR) apparatus that integrates biocatalytic and thermochemical technologies to convert woody biomass to products while utilizing residuals to produce thermal, dynamic and electrical power for an entire process.

3. The system of claim 1 wherein the system is a Distributed Biofuel Manufacturing System (DBMS).

4. The system of claim 1 wherein the biomass is at least one of a deliberate biomass and threat biomass.

5. The system of claim 1 wherein the integrated system utilizes plantations of trees to produce biofuels and wood pellets while performing decontamination.

6. The system of claim 5 wherein the plantations of trees comprise any combination of either of hybrid and transgenic poplar trees.

7. The system of claim 6 wherein the decontamination is made of soil.

8. The system of claim 6 wherein the decontamination is made of groundwater.

9. The system of claim 1 wherein the integrated system remediates threat biomass by onsite conversion of threat biomass into usable commercial products not limited to a usage of.

10. The system of claim 1 further comprising:
a Duel Use Pre-treatment Equipment apparatus including a pre-treatment standpipe which prepares any one of a hard biomass and a softwood biomass for conversion to biofuels.

11. A method, comprising:
converting a biomass utilizing a portable bio-refinery, co-developed catalysts and onsite biofuel and wood pellet production using an integrated system with an apparatus.

12. The method of claim 11 further comprising:
integrating biocatalytic and thermochemical technologies in a Portable Bio-Refinery (PBR) apparatus;
converting woody biomass to products; and
utilizing residuals to produce thermal, dynamic and electrical power for an entire process.

13. The method of claim 11 wherein the system is a Distributed Biofuel Manufacturing System (DBMS).

14. The method of claim 11 wherein the biomass is at least one of a deliberate biomass and threat biomass.

15. The method of claim 11 wherein the integrated system utilizes plantations of trees to produce biofuels and wood pellets while performing decontamination.

16. The method of claim 15 wherein the plantations of trees comprise any combination of either of hybrid and transgenic poplar trees.

17. The method of claim 16 wherein the decontamination is made of soil.

18. The method of claim 16 wherein the decontamination is made of groundwater.

19. The method of claim 11 wherein the integrated system remediates threat biomass by onsite conversion of threat biomass into usable commercial products not limited to a usage of.

20. The method of claim 11 further comprising:
preparing any one of a hard biomass and a softwood biomass for conversion to biofuel through a pre-treatment standpipe of a Duel Use Pre-treatment Equipment apparatus.
